(19) United States
(12) Patent Application Publication
(10) Pub. No.: US 2021/0087264 A1
(43) Pub. Date: Mar. 25, 2021

(54) ANTI-CLEVER-1 AGENTS FOR CONTROLLING LYSOSOMAL VATPASE PROTON PUMPS TO INCREASE LYSOSOMAL PH-VALUE AND THE CROSS-PRESENTATION OF DIGESTED MATTER

(71) Applicant: Faron Pharmaceuticals Oy, Turku (FI)

(72) Inventors: Maija-Leena HOLLMÉN, Piispanristi (FI); Miro VIITALA, Turku (FI); Jenna RANNIKKO, Turku (FI)

(73) Assignee: Faron Pharmaceuticals Oy, TURKU (FI)

(21) Appl. No.: 17/031,349

(22) Filed: Sep. 24, 2020

(30) Foreign Application Priority Data
Sep. 24, 2019 (FI) 20195804

(51) Int. Cl. C07K 16/24; A61P 43/00

(52) U.S. Cl. CPC C07K 16/24; A61K 2039/505; A61P 43/00

(57) ABSTRACT

A method of determining the efficacy of anti-Clever-1 therapy by measuring lysosomal pH, and a method of increasing lysosomal pH for improving an activation of the immune system in a patient, in which method an agent that inhibiting Clever-1 on the lysosomal proton pump (vATPase) is administered to a patient.

### FIELD OF THE INVENTION

The present invention relates to silencing or inhibiting CLEVER-1 in order to interact with lysosomal V0a1 proton pumps to increase lysosomal pH and antigen cross-presentation to the immune system for the clearance of diseases and enhancement of immune reactions, e.g. vaccination.

### BACKGROUND OF THE INVENTION

Macrophages engulf and digest debris, dying cells and un-wanted matter into lysosomes. Through the Golgi complex and endosomal maturation, the digested matter is then loaded on the MHC complex and normally cross-presented to the immune system as either being harmful and foreign if needed.

CLEVER-1 is a protein disclosed in the patent publication WO 03/057130, Common Lymphatic Endothelial and Vascular Endothelial Receptor-1. In recent years, increasing attention has been paid to the contribution of scavenger receptors in regulating macrophage responses to different stimuli. Clever-1 (also known as Stabilin-1) is a multifunctional molecule conferring scavenging ability on a subset of anti-inflammatory macrophages [1]. It is a binding protein that mediates adhesion of lymphocytes but also a scavenger receptor expressed on monocytes and macrophages. Recent publications indicate that Clever-1 also affects tumor growth and cancer progression [2], [3]. The proposed tumorigenic mechanisms have centered on the paradigm of Clever-1 as an adhesion and scavenger receptor.

### SUMMARY OF THE INVENTION

Despite Clever-1 function as an adhesion receptor, it has now been found that the majority of Clever-1 is located intracellularly within the endosomal recycling pathway and the trans-Golgi network where GGA adaptor proteins traffic Clever-1 between the trans-Golgi network and lysosomes. Therefore, it has been surprisingly found out that by targeting Clever-1 on monocytes and/or macrophages we can change lysosomal pH, which then determines endosomal maturation and cross-presentation of digested matter, i.e. it has been observed that through Clever-1 on macrophages and/or monocytes we can control lysosome proton pumps and affect the antigen (digested matter) presentation capability of Clever-1 positive monocytes and/or macrophages.

More detailed, by targeting Clever-1 on monocytes and/or macrophages we can directly alter the pH level of lysosomes through interacting with the vATPase proton pump subunit V0a1, by which we can prevent the quick degradation of macrophage digested matter and thereby support endosomal maturation and the cross-presentation of digested matter.

The findings of the present invention provide use of an agent capable of inhibiting Clever-1 on the lysosomal proton pump (vATPase) to increase lysosomal pH and antigen (digested matter) cross-presentation and thereby activating the immune system of an individual. Therefore, the present invention provides a method of increasing lysosomal pH for improving an activation of the immune system in a patient, said method comprising
administering an agent that inhibiting Clever-1 on the lysosomal proton pump (vATPase) to said patient.

Further, the present invention provides a method of determining the efficacy of anti-Clever-1 therapy, when an agent that inhibiting Clever-1 on the lysosomal proton pump (vATPase) is administered in a patient, said method comprising
obtaining monocytes from a blood sample drawn from the patient prior to the administration of said agent,
obtaining monocytes from a blood sample drawn from the patient after the administration of said agent, and
measuring lysosomal pH from said monocytes and comparing the measured lysosomal pH values measured prior to the administration and after the administration of said agent, wherein an increased level of the lysosomal pH is an indication of the inhibition of Clever-1 acting on the lysosomal proton pump (vATPase) and efficiency of the anti-Clever.1 therapy.

The improved antigen presentation according to the present invention can be utilized in the treatment of the diseases or conditions related to lysosome function and the immune system. Hence, the present invention provides an agent capable of inhibiting Clever-1 on the lysosomal proton pump (vATPase) in an individual for use in an activation of the immune system, wherein the improved action can be observed e.g. as an increase of the lysosomal pH. According to an embodiment of the present invention an agent capable of inhibiting Clever-1 on the lysosomal proton pump (vATPase) in an individual can be used in the treatment of lysosomal disorders. An improved activation achieved by an agent inhibiting Clever-1 can also be utilized in the treatment of chronic infections, and in the enhancements of immune reactions, e.g. by vaccination.

### DESCRIPTION OF THE DRAWINGS

The invention will be described in more detail with reference to appended drawings, in which

FIG. 1. A) acLDL endosomal internalization is significantly decreased in Clever-1 silenced macrophages. B) Lysosomal pH is significantly increased in Clever-1 silenced macrophages. C) acLDL containing endosomal pH is significantly increased in Clever-1 silenced macrophages.

FIG. 2. Acetylated LDL induced translocation of Clever-1 into Lamp1+ lysosomes.

FIG. 3. Primary human macrophages were treated with either anti-Clever-1 antibody 9-11 or anti-Clever-1 antibody FP-1305. Macrophage lysosome pH was measured using Lysosensor 4 h and 24 hours after treatment. The results show that FP-1305 normalizes lysosomal pH, while 9-11 doesn't.

### DETAILED DESCRIPTION OF THE INVENTION

As shown in the Experimental part of the present disclosure, our data shows that Clever-1 interacts directly with the vacuolar ATPase (v-ATPase) subunit V0a1. V-ATPase is a multi-subunit protein complex composed of the cytosolic V₁ and lysosomal membrane-anchored V₀ sections that reversibly assemble into a functional proton pump and decrease lysosomal pH. Lysosomes are the primary organelles for intracellular degradation, and lysosomal enzymes require low pH to function optimally. The lysosomal V0 sector of v-ATPase consists of six subunits (a, c, c″, d, e and Ac45 in mammals), of which the subunit V0a has four tissue- and cell-specific isoforms V0a1-4. V0a1 is expressed by macrophages and cancer cell lines. Recently, it has been reported that misrouting of the V0a1 subunit from lysosomes to the endosomal recycling pathway disrupts the assembly of functional v-ATPase and therefore increases lysosomal pH, thus preventing the degradation of lysosomal cargo [4]. Significantly, we have now observed that the interaction of Clever-1 with V0a1 offers an intriguing mechanistic link between macrophage immunometabolism, phagosomal maturation and cross-presentation of digested matter. We have now found that the inhibition of Clever-1 on monocytes and/or macrophages prevents the interactions with vacuolar ATPase (v-ATPase) subunit V0a1 to decrease lysosome pH value, which then leads to endosomal maturation and MHC complex loading of the digested matter instead of degradation without cross-presenting the digested matter to the immune system. Thus, by inhibiting Clever-1 we can now control lysosome proton pumps and change the antigen (digested matter) cross-presentation capability of Clever-1 positive monocytes/macrophages and thereby activating the immune system in an individual.

According to an embodiment of the present invention, an agent capable of inhibiting Clever-1 on the lysosomal proton pump (vATPase) can be used in an activation of the immune system in an individual to increase lysosomal pH and to improve antigen (digested matter) cross-presentation. A method according to an embodiment of the present invention of increasing lysosomal pH for improving an activation of the immune system in a patient comprises
administering an agent that inhibiting Clever-1 on the lysosomal proton pump (vATPase) to said patient.

An activation of the immune system according to the present invention based on the method for controlling lysosomal vATPase proton pumps to increase lysosomal pH-value and thereby improving antigen cross-presentation.

According to an embodiment of the present invention, an activation of the immune system may be verified by measuring lysosomal pH from monocytes/macrophages obtained from a blood sample drawn from the patient. In a typical embodiment of the present invention, lysosomal pH is measured from monocytes obtained from a blood sample drawn from the patient prior to the administration of an agent capable of inhibiting Clever-1 and monocytes obtained from a blood sample drawn from the patient after the administration of an agent capable of inhibiting Clever-1. The measured pH values are compared, and an increased level of the lysosomal pH is an indication of the inhibition of Clever-1 acting on the lysosomal proton pump (vATPase) and efficiency of the anti-Clever-1 therapy.

A method according to an embodiment of the present invention of determining the efficacy of anti-Clever-1 therapy, when an agent capable of inhibiting Clever-1 on the lysosomal proton pump (vATPase) is administered in a patient, comprises
obtaining monocytes from a blood sample drawn from the patient prior to the administration of anti-Clever-1 agent,
obtaining monocytes from a blood sample drawn from the patient after the administration of anti-Clever-1 agent, and
measuring lysosomal pH from said monocytes/macrophages and comparing the measured lysosomal pH values, wherein an increased level of the lysosomal pH is an indication of the inhibition of Clever-1 acting on the lysosomal proton pump (vATPase) and efficiency of the anti-Clever-1 therapy.

Consequently, the increased level of endosomal pH may be used as a marker for monitoring treatment response in an individual. The endosomal pH may be determined from the blood monocytes obtained from the blood drawn from a patient. An endosomal pH value measured may be used as a marker for the patient response to the treatment comprising administering an anti-Clever-1 agent(s), when the pH value is compared to pH value measured from the same patient before administering an anti-Clever-1 agent(s) in the patient or the pH values of one or more previous measurements carried out at different time points in the same patient.

Determining of the endosomal pH from monocytes obtained from a blood sample drawn from the patient can be carried out by commonly known methods, for example by using commercial LysoSensor™ (ThermoFisher Scientific) probes, i.e. fluorescent pH indicators. The LysoSensor™ reagents exhibit a pH-dependent decrease in fluorescence intensity upon acidification and therefore it can be used for measuring immune activation according to the present invention.

According to an exemplary embodiment of the present invention, when the increased level of the pH value is at least 0.5 unit between the measurements prior to administration of anti-Clever-1 agent(s) and after the administration of anti-Clever-1 agent(s), it is an indication of the efficiency of the anti-Clever-1 therapy.

According to the present invention an agent capable of inhibiting Clever-1 may be any suitable agent that inhibiting Clever-1 and that exhibit the desired activity. According to an embodiment of the present invention, an agent inhibiting Clever-1 comprises an antibody or fragment(s) thereof capable of inhibiting Clever-1. The term “an antibody or fragment(s) thereof” is used in the broadest sense to cover an antibody or fragment(s) thereof which are capable to bind CLEVER-1 molecule in an individual. Especially, it shall be understood to include chimeric, humanized or primatized antibodies, as well as antibody fragments and single chain antibodies (e.g. Fab, Fv), so long they exhibit the desired biological activities.

According to an embodiment of the invention, an anti-CLEVER-1 antibody is a humanized monoclonal anti-CLEVER-1 antibody. In an embodiment according to the present invention, an anti-CLEVER-1 antibody may be a humanized antibody based on the monoclonal antibody 3-372 disclosed in the patent publication WO 03/057130. According to an embodiment of the present invention an anti-CLEVER-1 antibody is a humanized monoclonal CLEVER-1 antibody, previously presented in the patent publication WO2017/182705. In an embodiment of the present invention, the anti-CLEVER-1 antibody is a humanized monoclonal immunoglobulin G4_{K} antibody bexmarilimab (proposed International Nonproprietary Name (INN)) as disclosed in WHO Drug Information, Vol. 33, No. 4, pages 814-815 (2019)), or bexmarilimab variant or the antibody in a bexmarilimab biosimilar.

Anti-CLEVER-1 antibody bexmarilimab is an exemplary antibody used in the method according to the present invention. As used herein, “bexmarilimab” means the IgG4 monoclonal antibody with the structure described in WHO Drug Information, Vol. 33, No. 4, pages 814-815 (2019).

A bexmarilimab biosimilar means a biological product which is approved by a regulatory agency in any country for marketing as a bexmarilimab biosimilar. In an embodiment, a bexmarilimab biosimilar comprises a bexmarilimab variant as the drug substance. In an embodiment, a bexmarilimab biosimilar has substantially the same amino add sequence of heavy and light chains as bexmarilimab. As used herein, a “bexmarilimab variant” means an antibody which comprises sequences of heavy chain and light chain that are identical to those in bexmarilimab, except for having one or more conservative amino acid substitutions at positions that are located outside of the light chain CDRs and/or one or more conservative amino acid substitutions that are located outside of the heavy chain CDRs, e.g. the variant positions are located in the framework regions or the constant region. In other words, bexmarilimab and a bexmarilimab variant comprise identical CDR sequences, but differ from each other due to having a conservative amino add substitution at other positions in their full-length light and heavy chain sequences. A bexmarilimab variant is substantially the same as bexmarilimab with respect to binding affinity to CLEVER-1.

According to an embodiment of the present invention, a cell line producing the anti-CLEVER-1 antibody bexmarilimab has been deposited on 27 May 2020 under the terms of the Budapest Treaty on the International Recognition of the Deposit of Micro-organisms for the Purposes of Patent Procedure with the DSMZ-German Collection of Microorganisms and Cell Cultures GmbH, Inhoffenstrasse 7B, D-38124 Braunschweig, Germany, and has the accession number DSM ACC3361. The present invention is not to be limited in scope by the culture deposited, since the deposited embodiment is intended as a single illustration of one aspect of the invention and any culture that is functionally equivalent is within the scope of this invention. The deposit of material herein does not constitute an admission that the written description herein contained is inadequate to enable the practice of any aspect of the invention, including the best mode thereof, nor is it to be construed as limiting the scope of the claims to the specific illustration that it represents.

We believe that the method of activating immune system according to the present invention is useful in the treatment of the diseases or conditions related to the function of the immune system. An agent inhibiting Clever-1 can be utilized in the treatment of the diseases or conditions related to lysosome function and the immune system, wherein the disease or condition is selected from the group comprising of lysosomal disorders, chronic infections and cancer. A method according to the present invention for treatment of lysosomal disorders, chronic infections and/or cancer comprises an administration of an agent that inhibiting Clever-1 on the lysosomal proton pump (vATPase) to a patient Further, an improved activation of the immune system achieved by an agent inhibiting Clever-1 can be utilized in the enhancements of immune reactions, e.g. by vaccination. According to the present invention, an agent inhibiting Clever-1 can be used for an adjuvant of a vaccine, wherein through the increased endosomal pH, the activation of the immune system may be improved.

In the present disclosure, the term “patient” or “individual” refers to a human.

The term “treatment” or “treating” shall be understood to include complete curing of a disease as well as amelioration or alleviation of said disease.

A further embodiment of the present invention is directed to a pharmaceutical composition comprising an agent capable of inhibiting Clever-1, preferably an antibody or fragment(s) thereof capable of inhibiting Clever-1, and an appropriate excipient for use in in the treatment of the diseases or conditions related to lysosome function and the immune system. The pharmaceutical compositions to be used in the present invention can be administered by any means that achieve their intended purpose. For example, administration can be intravenous, intraarticular, intra-tumoural or subcutaneous. In addition to the pharmacologically active compounds, the pharmaceutical preparations of the compounds preferably contain suitable pharmaceutically acceptable carriers comprising excipients and auxiliaries that facilitate processing of the active compounds into preparations that can be used pharmaceutically.

### EXPERIMENTAL

Example 1: Clever-1 Regulates Lysosome pH and Endosomal Maturation Clever-1 siRNA-transfected KG-1 macrophages were pulsed with 10 μg/ml of

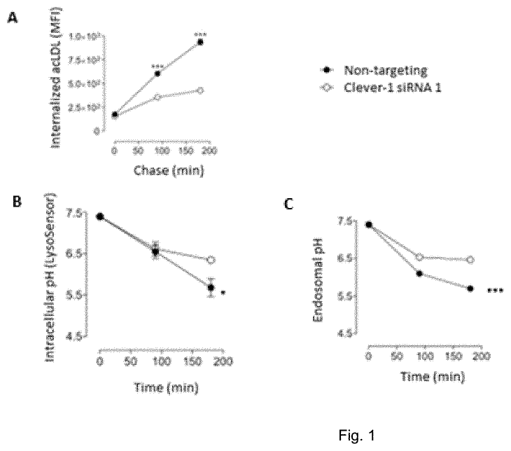

Alexa Fluor 488-labelled acLDL, 1 μM of LysoSensor Green DND-189 detecting lysosomal pH or 10 μg/ml of pHrodo Green-labelled acLDL detecting acLDL containing endosomal pH, at 16° C. for 30 min and incubated at 37° C. for the indicated chase periods before flow cytometric analysis. Extracellular fluorescence was quenched with 0.1% trypan blue. Experiments showed that significantly lower levels of acLDL are degraded and internalized in Clever-1 silenced macrophages compared to wild type macrophages and that lysosomal pH (Lysosensor) and acLDL carrying endosomal pH was significantly increased in Clever-1 siRNA silenced macrophages (FIG. 1).

### Example 2: Acetylated LDL Induced Translocation of Clever-1 Into Lamp1+ Lysosomes

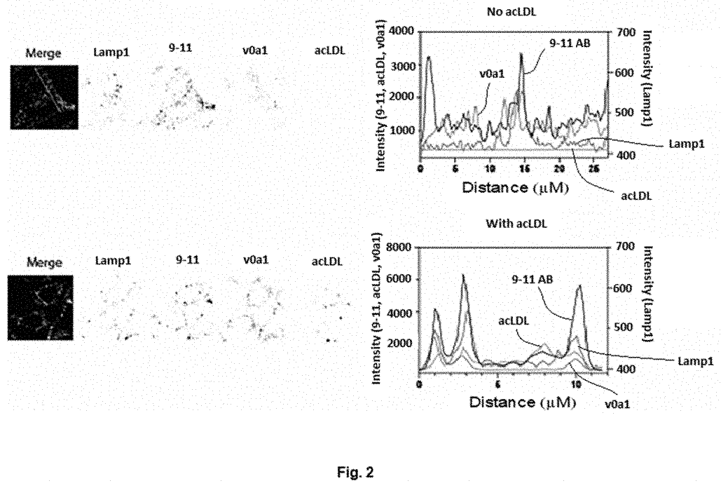

KG-1 cells were cultured in Iscove's modified Dulbecco's medium (IMDM) supplemented with 20% FCS and penicillin-streptomycin. 10⁵ cells/well were plated on 8-well ibiTreat μ-Slide chambers (ibidi, cat. 80826) and treated with 300 nM PMA (Sigma, cat. P1585) for three days to induce macrophage differentiation. On day 4, the differentiated cells were fed with or without 10 μg/ml acLDL-alexa647 for 3 hours. The cells were fixed in 4% paraformaldehyde and permeabilized and blocked in 0.1% triton X-100, 5% goat serum and 1:1000 Kiovig (LE-072213, Baxter) in PBS. The cells were stained for Lamp1 (1:100, Cell signalling, cat. 9091), v0a1 (1:100, Abnova, cat. H00000535-A01) and Clever-1 (10 μg/ml, 9-11, InVivo Biotech). The primary antibodies were incubated for 1 h at room temperature and the secondary antibodies (1:300 ThermoFisher, A11006, A31556, A11030) for 30 minutes at room temperature. Images were obtained with Marianas spinning disk (Intelligent Imaging Innovations) connected to CSU-W1 scanning unit (Yokogawa) and Orca Flash 4 sCMOS camera (Hamatsu) using a 100× objective (oil, NA 1.4, Plan-Apochromat, Carl Zeiss). Images were analysed for co-localization using ImageJ software. The results are shown in FIG. 2.

Without ligand (upper panel of FIG. 2) Clever-1 is mainly located in Lamp1 negative endosomal structures. The scavenging of acLDL induces rapid translocation of Clever-1 into Lamp1 positive lysosomes together with v0a1 (lower panel of FIG. 2), a component of the vacuole-ATPase complex inducing acidification of lysosomal vesicles.

### Example 3: Anti-Clever-1 Antibody FP-1305 Normalizes Lysosomal pH

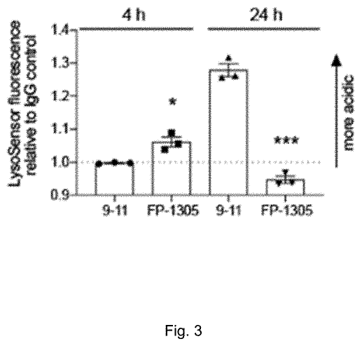

Primary human macrophages were treated with either anti-Clever-1 antibody 9-11 or anti-Clever-1 antibody FP-1305 (DSM ACC3361). In detail, M2-polarized buffy-coat-derived macrophages (100 μM dexamethasone+20 ng/ml IL-4, 24 h) were treated with the indicated antibodies at 50 μg/ml for the indicated time points. LysoSensor Green dye was added for the final 4 h of incubation. Cells were washed once with PBS+5 mM EDTA, collected by scraping and analyzed by FACS with 7-AAD as viability dye. Macrophage lysosome pH was measured using Lysosensor 4 h and 24 hours after incubation. The results are shown in FIG. 3. FP-1305 normalizes lysosomal pH, while 9-11 doesn't but the lysosomal pH is more acidic which induces rapid degradation of antigens.

### CITED REFERENCES

[1] Kzhyshkowska J., Gratchev A., Goerdt S., Stabilin-1, a homeostatic scavenger receptor with multiple functions. J Cell Mol Med 2006;10(3):635-49.

[2] Karikoski M., Marttila-lchihara F., Elima K., Rantakari P., Hollmén M., Kelkka T., et al., Clever-1/Stabilin-1 Controls Cancer Growth and Metastasis. Clinical Cancer Research 2014,20(24):6452-64 doi 10.1158/1078-0432.CCR-14-1236.

[3] Viitala M., Virtakoivu R., Tadauon S., Rannikko J., Jalkanen S., Hollmén M., Immunotherapeutic Blockade of Macrophage Clever-1 Reactivates the CD8+ T-cell Response against Immunosuppressive Tumors. Clin Canc Res. February 2019 doi: 10.1158/1078-0432

[4] Bagh M., Peng S., Chandral G., Zhang Z., Singh S., Pattabiraman N., Liu A. & Anil B., Mukherjee A. B. Misrouting of v-ATPase subunit V0a1 dysregulates lysosomal acidification in a neurodegenerative lysosomal storage disease model. Nature Communications 2017;8:14612 doi: 10.1038

1. A method of increasing lysosomal pH for improving an activation of the immune system in a patient, said method comprising
administering an agent that inhibiting Clever-1 on the lysosomal proton pump (vATPase) to said patient.

2. The method according to claim 1, wherein the anti-Clever-1 agent comprises an anti-Clever-1 antibody or fragment(s) thereof.

3. The method according to claim 2, wherein an anti-CLEVER-1 antibody is bexmarilimab (proposed INN) described in WHO Drug Information, Vol. 33, No. 4, pages 814-815 (2019) or bexmarilimab variant or the antibody in a bexmarilimab biosimilar.

4. The method according to claim 2, wherein an anti-CLEVER-1 antibody is antibody FP-1305 (DSM ACC3361).

5. A method of determining the efficacy of anti-Clever-1 therapy comprising an administration an agent capable of inhibiting Clever-1 to a patient, said method comprising
obtaining monocytes from a blood sample drawn from the patient,
administering an agent capable of inhibiting CLEVER-1 to a patient,
obtaining monocytes from a blood sample drawn from the patient after the administration of said agent, and
measuring lysosomal pH from said monocytes obtained prior to and after the administration of said agent and comparing the measured lysosomal pH values , wherein an increased level of the lysosomal pH is an indication of the efficacy of the anti-Clever-1 therapy.

6. The method according to claim 5, wherein the anti-Clever-1 agent comprises an anti-Clever-1 antibody or fragment(s) thereof.

7. The method according to claim 6, wherein an anti-CLEVER-1 antibody is bexmarilimab (proposed INN) described in WHO Drug Information, Vol. 33, No. 4, pages 814-815 (2019) or bexmarilimab variant or the antibody in a bexmarilimab biosimilar.

8. The method according to claim 6, wherein an anti-CLEVER-1 antibody is antibody FP-1305 (DSM ACC3361).

9. The method according to claim 5, wherein the increased level of the pH value is at least 0.5 unit.

10. An agent capable of inhibiting Clever-1 for use in the treatment of the diseases or conditions related to lysosome function and the immune system, wherein an improved activation of the immune system is observed by an increase of the lysosomal pH.

11. The agent according to claim 10 for use in treatment of the diseases or conditions related to lysosome function and the immune system, wherein the agent comprises an antibody or fragment(s) thereof capable of inhibiting Clever-1.

12. The agent according to claim 10 for use in treatment of the diseases or conditions related to lysosome function and the immune system, wherein the disease or condition is selected from the group comprising of lysosomal disorders, chronic infections and cancer.

13. The agent according to claim 10 for use in treatment of the diseases or conditions related to lysosome function and the immune system, wherein the agent is used as an adjuvant of the vaccine.

14. The agent according to claim 10 for use in treatment of the diseases or conditions related to lysosome function and the immune system, wherein an anti-CLEVER-1 antibody is bexmarilimab (proposed INN) described in WHO Drug Information, Vol. 33, No. 4, pages 814-815 (2019) or bexmarilimab variant or the antibody in a bexmarilimab biosimilar.

15. The agent according to claim 10 for use in treatment of the diseases or conditions related to lysosome function and the immune system, wherein an anti-CLEVER-1 antibody is antibody FP-1305 (DSM ACC3361).
